(19) United States
(12) Patent Application Publication
(10) Pub. No.: US 2020/0017900 A1
(43) Pub. Date: Jan. 16, 2020

(54) GENETIC MARKERS FOR DISTINGUISHING THE PHENOTYPE OF A CANNABIS SATIVA SAMPLE

(71) Applicants: Ilaria Boschi, Roma (IT); Fidelia Cascini, Roma (IT); Jamila Bernardi, Pontenure (IT); Laura Baldassarri, Roma (IT); Alessio Farcomeni, Roma (IT)

(72) Inventors: Ilaria Boschi, Roma (IT); Fidelia Cascini, Roma (IT); Jamila Bernardi, Pontenure (IT); Laura Baldassarri, Roma (IT); Alessio Farcomeni, Roma (IT)

(21) Appl. No.: 16/343,480

(22) PCT Filed: Oct. 21, 2016
(86) PCT No.: PCT/EP2016/075403

§ 371 Date: Apr. 19, 2019

(51) Int. Cl. C12Q 1/686; C12Q 1/6806

(52) U.S. Cl. CPC C12Q 1/686; C12Q 1/6806; C12Q 2600/16; C12Q 2600/13; C12Q 2600/156

(57) ABSTRACT

The present invention concerns the field of molecular markers suitable for distinguishing marijuana from hemp and the generation of tools for forensic medicine and pharmaceutical research. The invention further provides uses of the molecular markers and methods for distinguishing marijuana from hemp samples as well as a kit.

### FIELD OF THE INVENTION

The present invention concerns the field of molecular markers suitable for distinguishing marijuana from hemp and the generation of tools for forensic medicine and pharmaceutical research. The invention further provides uses of the molecular markers and methods for distinguishing marijuana from hemp samples as well as a kit.

### STATE OF THE ART

Cannabis sativa L. (commonly called cannabis) is a herbaceous plant belonging to the Cannabis genus, family of Cannabaceae.

The Cannabis genus includes wild and cultivated forms that are morphologically variable. Controversy over the taxonomic organization still remains: some authors have proposed a monotypic genus, C. sativa, while others have argued that Cannabis is composed of two species, C. sativa and C. indica, and some have included a third species, C. ruderalis, in the genus. (Hillig, 2005).

Beside this taxonomic uncertainty, the species C. sativa L. includes varieties suitable for recreational and therapeutic purposes (commonly named marijuana or simply cannabis) as well as varieties appropriate for industrial use only (usually named hemp). This feature depends on the capability of each cannabis variety or strain, to synthesize and accumulate secondary metabolites known as cannabinoids. Cannabinoids represent a group of more than 100 natural products within which tetrahydrocannabinolic acid (THCA) is the main (psycho)active compound.

Tethraydrocannabinolic acid (THCA) and cannabidiolic acid (CBDA) are from the same precursor cannabigerolic acid (CBGA). THCA-synthase enzyme or CBDA-synthase enzyme are respectively responsible for the synthesis of THCA or CBDA.

Information is currently available regarding the genetics of cannabis, which has a diploid genome with a karyotype composed of nine autosomes and a pair of sex chromosomes, as well as for the sequencing of the principal genes involved in the cannabinoids biosynthetic pathway (THCA synthase and cannabidiolic acid synthase genes).

The meaning of many different lines of research is reasonably twofold: to better understand genetic mechanisms regulating chemical properties of the plant (explaining both its toxic effects and its therapeutic applications) and to offer tools suitable for forensic investigations to contrast the illegal market at the same time protect the economy related to the industrial destination of hemp.

Despite all the efforts to establish genetic relationships as well as to highlight genetic differences among plant varieties (with different chemical phenotypes and different psychoactive effects), this topic has remained until now a challenge for the scientific community particularly concerning the most investigated genes of cannabis to date, concerning key enzymes in cannabinoids biosynthesis THCA synthase (THCAS) and cannabidiolic acid synthase (CBDAS).

The gene coding for the enzyme THCA-synthase, that is responsible for the production of THC from the CBG precursor, has been identified (Taura, 1995; Sirikantaramas et al., 2004).

Several functional and nonfunctional sequence variants of this gene have been published and sequence polymorphisms have been employed for marker application. Kojoma et al. (2006) published 13 different strains of cannabis plants distinguishing, by implementing a specific PCR marker, high-THC (drug-type) from low/absent-THC (fiber-type) varieties.

Rotherham & Harbison (2011) developed a single nucleotide polymorphism (SNP) assay, based on 4 polymorphisms of THCA synthase gene for the differentiation of drug and non-drug cannabis plants.

The sequence of CBDA synthase gene, resulted very similar to that of the THCA synthase gene (homology 87.9%) (Yoshikai, Taura, Morimoto & Shoyama, 2001), but the variability between the sequences has still to be been dealt with in depth.

The need and importance is increasingly felt for simple biotechnological assays which allow to distinguish the phenotype to the plant, even before it grows to maturity.

It is therefore object of the present invention the development of molecular markers and a kit suitable for the generation of tools for forensic research and pharmaceutical applications.

### SUMMARY OF THE INVENTION

The problem underlying the present invention is that of making available methods for allowing the identification of the phenotype of a cannabis plant.

This problem is solved by the present finding by the use of genetic markers, and in particular:
33 major nucleotide substitutions (SNPs) of THCAS and CBDAS genes were detected in the alignment of the sequences from high-THC type (drug-type) and low/absent-THC type (fiber-type) strains;
a deletion of four bases, position 153-156 (CGTA), in high-THC type (drug-type) strains and an insertion of three bases, in position 755 (AAC), in low/absent-THC type (fiber-type) strains, were detected in CBDA synthase gene.

It appeared that the nucleotide substitutions (SNPs) and the deletion/insertion have a correlation with the reduction of THCA production in cannabis plants.

The present invention concerns the use of specific genetic markers for the discrimination/identification of the fiber-type variety from the drug-type variety of Cannabis sativa, wherein said genetic markers are:
SNPs of the CBDAS gene chosen from the group consisting of: “pos407”, “pos545”, “pos583”, “pos588”, “pos613”, “pos637”, “pos688” and “pos704”,
SNPs of the THCAS gene chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”;
deletion of four bases, position 153-156, and insertion of three bases, in position 755+3 in the CBDAS gene of high-THC type (drug-type) strains.

SNPs identified in THCA synthase gene are numbered referring to THCAS coding sequence of the drug-type cultivar Skunk (KJ469378); SNPs and deletion/insertion in CBDA synthase gene are numbered referring to CBDAS coding sequence of fiber-type cultivar Carmen (KJ469374).

For the purposes of the present invention, each genetic marker can be identified by the nucleotide position in the CBDAS gene or in the THCAS gene, and indicated for example as “pos417” or position 417 or locus 417. All the definitions are interchangeable. Furthermore:
deletion of four bases, position 153-156 in the CBDAS gene, means that bases 153, 154, 155 and 156 of the CBDAS gene of high-THC type (drug-type) strains are deleted; and
insertion of three bases of the CBDAS gene in position 755+3 means that three bases: AAC, are inserted from position 755 of the CBDAS gene of high-THC type (drug-type) strains, in particular A in position 756, A in position 757 and C in position 758.

As will be further described in the detailed description of the invention, the molecular markers of the invention have the advantages of being specific either for the CBDAS or the THCAS gene and of having an absolute diagnostic value with a 100% certainty of success.

A further advantage of the molecular markers according to the present invention is the fact that it is possible to distinguish between the fiber-type and the drug-type varieties by using only one single marker.

A still further advantage of the present molecular marker is that the absolute diagnostic value between the fiber-type and the drug-type varieties can be obtained starting from any part of the plant, or from the seed.

A further aspect of the present invention is the use of the genetic markers, for distinguishing a sample of the fiber-type variety of Cannabis sativa from the drug-type variety.

According to another aspect, the described invention provides a method for discriminating the fiber-type variety from the drug-type variety of Cannabis sativa, comprising the steps of
a. providing a sample from a Cannabis sativa plant or seed;
b. extracting the DNA from said sample;
c. conducting a PCR on the PCR sample of step b.;
d. sequencing the PCR product of step c.;
e. analyzing the sequence of PCR product by electrophoresis;
f. identifying at least one of the SNPs or deletions of said PCR product wherein,
a SNP of the CBDAS gene is chosen from the group consisting of: “pos407”, “pos545”, “pos583”, “pos588”, “pos613”, “pos637”, “pos688” and “pos704”;
a SNP of the THCAS gene is chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”;
deletion of four bases, position 153-156, and insertion of three bases, AAC, in position 755+3 in the CBDAS gene of high-THC type (drug-type) strains;

and wherein,

when the SNP of the CBDAS gene is:

G in position 407, the sample is a fiber-type variety;

A in position 407, the sample is a drug-variety;

G in position 545, the sample is a fiber-type variety;

C in position 545, the sample is a drug-variety;

A in position 583, the sample is a fiber-type variety;

C in position 583, the sample is a drug-variety;

T in position 583, the sample is a drug-variety;

C in position 588, the sample is a fiber-type variety;

T in position 588, the sample is a drug-variety;

A in position 613, the sample is a fiber-type variety;

G in position 613, the sample is a drug-variety;

C in position 637, the sample is a fiber-type variety;

G in position 637, the sample is a drug-variety;

T in position 688, the sample is a fiber-type variety;

A in position 688, the sample is a drug-variety;

C in position 704, the sample is a fiber-type variety;

G in position 704, the sample is a drug-variety;

when the SNP of the THCAS gene is:

C in position 136, the sample is a fiber-type variety;

G in position 136, the sample is a drug-variety;

C in position 137, the sample is a fiber-type variety;

T in position 137, the sample is a drug-variety;

A in position 154, the sample is a fiber-type variety;

G in position 154, the sample is a drug-variety;

C in position 221, the sample is a fiber-type variety;

T in position 221, the sample is a drug-variety;

T in position 269, the sample is a fiber-type variety;

A in position 269, the sample is a drug-variety;

G in position 287, the sample is a fiber-type variety;

C in position 287, the sample is a drug-variety;

C in position 300, the sample is a fiber-type variety;

T in position 300, the sample is a drug-variety;

T in position 355, the sample is a fiber-type variety;

A in position 355, the sample is a drug-variety;

C in position 383, the sample is a fiber-type variety;

T in position 383, the sample is a drug-variety;

A in position 385, the sample is a fiber-type variety;

G in position 385, the sample is a drug-variety;

A in position 409, the sample is a fiber-type variety;

T in position 409, the sample is a drug-variety;

G in position 412, the sample is a fiber-type variety;

A in position 412, the sample is a drug-variety;

G in position 418, the sample is a fiber-type variety;

A in position 418, the sample is a drug-variety;

A in position 424, the sample is a fiber-type variety;

G in position 424, the sample is a drug-variety;

T in position 494, the sample is a fiber-type variety;

A in position 494, the sample is a drug-variety;

T in position 505, the sample is a fiber-type variety;

C in position 505, the sample is a drug-variety;

C in position 612, the sample is a fiber-type variety;

T in position 612, the sample is a drug-variety;

A in position 678, the sample is a fiber-type variety;

G in position 678, the sample is a drug-variety;

A in position 699, the sample is a fiber-type variety;

T in position 699, the sample is a drug-variety;

T in position 744, the sample is a fiber-type variety;

G in position 744, the sample is a drug-variety;

T in position 749, the sample is a fiber-type variety;

A in position 749, the sample is a drug-variety;

G in position 763, the sample is a fiber-type variety;

T in position 763, the sample is a drug-variety;

A in position 862, the sample is a fiber-type variety;

G in position 862, the sample is a drug-variety;

G in position 864, the sample is a fiber-type variety;

A in position 864, the sample is a drug-variety;

C in position 869, the sample is a fiber-type variety;

T in position 869, the sample is a drug-variety.

In a further aspect the invention provides a kit for distinguishing between the fiber-type variety and the drug-type variety of Cannabis sativa by using one or more genetic markers chosen from the group consisting of:
SNPs of the CBDAS gene chosen from the group consisting of: “pos407”, “pos545”, “pos583”, “pos588”, “pos613”, “pos637”, “pos688” and “pos704”,
SNPs of the THCAS gene chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases, position 153-156, and insertion of three bases, AAC, in position 755+3 in the CBDAS gene of high-THC type (drug-type) strains

said kit comprising one or more sets of primers and/or probes and an instructions leaflet.

The present kit may be used according to the method of the invention (direct sequencing of genes) and also using other methods such as real-time PCR, any kind of electrophoresis, SNaPshot (SNPs only), and microchip, and may comprise further components necessary for DNA extraction from any plant sample or seed.

### BRIEF DESCRIPTION OF THE DRAWINGS

The characteristics and advantages of the present invention will be apparent from the detailed description reported below, from the Examples, and from the annexed FIGS. 1-3, wherein:

FIG. 1. Single Nucleotide Polymorphisms identified in THCA synthase and CBDA synthase genes. 47 THCA synthase (FIG. 1A)+40 CBDA synthase (FIG. 1B) genes different genetic loci (SNPs) have been independently identified as discriminating between fiber-type and drug-type cannabis varieties.

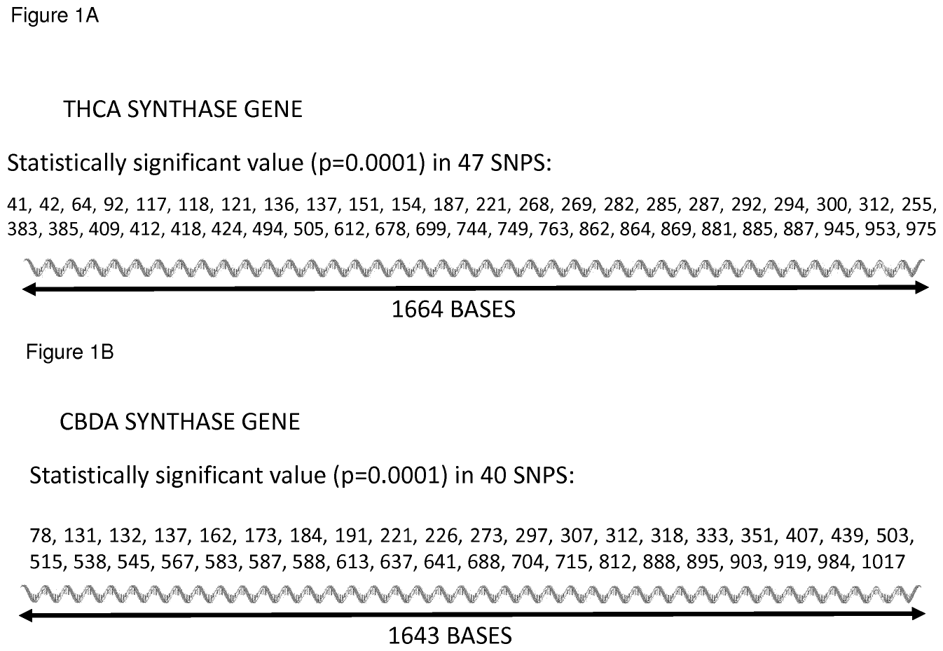

FIG. 2. Deletion of four bases, position 153-156, and insertion of three bases, in position 755+3 in the CBDAS gene of high-THC type (drug-type) strains.

FIG. 3. Box plot showing the score for fiber-type and drug-type plants, based on the SNPs and deletions identified, score (d).

FIG. 4. ROC curve for the THCA and CBDS score for fiber-type and drug-type plants, based on the SNPs identified, score (d).

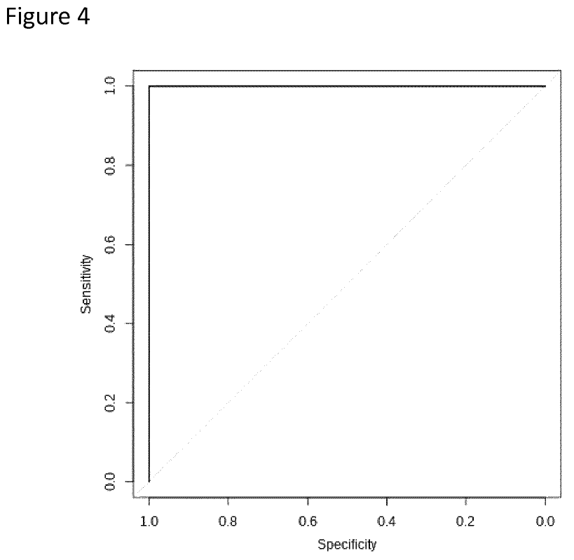

### DETAILED DESCRIPTION OF THE INVENTION

The present invention concerns a genetic marker for the discrimination/identification of the fiber-type variety from the drug-type variety of Cannabis sativa, wherein said genetic markers are:
SNPs of the CBDAS gene chosen from the group consisting of: “pos407”, “pos545”, “pos583”, “pos588”, “pos613”, “pos637”, “pos688” and “pos704”,
SNPs of the THCAS gene chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”;

and
deletion of four bases, position 153-156, and insertion of three bases, AAC, in position 755+3. in the CBDAS gene of high-THC type (drug-type) strains.

The genetic marker of the present invention is preferably chosen from the group consisting of a SNP of THCAS or CBDAS genes, or a deletion/insertion of the CBDAS gene.

By the term “SNP” as used herein is intended a “single nucleotide polymorphism”, or a variation in a single nucleotide that occurs at a specific position in the genome, where each variation is present to some appreciable degree within a population (e.g. >1%).

The molecular markers of the invention have the advantages of being specific either for the CBDAS or the THCAS gene and of having an absolute diagnostic value with a 100% certainty of success.

A further advantage of the molecular markers according to the present invention is the fact that it is possible to distinguish between the fiber-type and the drug-type varieties by using only one single marker.

The desired test needed to allow to distinguish between varieties prior to the stage of plant maturity when the synthesis and the storage of cannabinoids start.

In fact a still further advantage of the present molecular marker is that the absolute diagnostic value between the fiber-type and the drug-type varieties can be obtained starting from any part of the plant, and especially starting from the seed. In this way a seed can be enough to distinguish if the two plant types.

The genetic markers of the present invention have been developed on two experimental cultivations of cannabis (marijuana and hemp), with the aim of finding significant differences among the two sub-groups of varieties (fiber-type and drug-type sub-groups) to design a reliable diagnostic test, which is cheap, fast and easy to be used for industrial applications as well as for forensic investigations.

The test is based on THCA-synthase and CBDA-synthase genetic markers after comparing chemical and genetic features of varieties belonging to the two different sub-groups.

A further aspect of the present invention is thus the use of the genetic markers, for distinguishing a sample of the fiber-type variety of Cannabis sativa from the drug-type variety. In a preferred aspect, the invention provides the use of the genetic markers for identifying a fiber-type variety of Cannabis sativa sample from the drug-type variety sample, wherein said sample any part of the plant, and in particular said sample is chosen from the group consisting of seeds, inflorescences (or flowers), leaves, roots, nodes, stem or stalk.

According to another aspect, the described invention provides a method for discriminating the fiber-type variety from the drug-type variety of Cannabis sativa, comprising the steps of
a. providing a sample from a Cannabis sativa plant or seed;
b. extracting the DNA from said sample;
c. conducting a PCR on the PCR sample of step b.;
d. sequencing the PCR product of step c.;
e. analyzing the sequence of said PCR product by electrophoresis;
f. identifying at least one of the SNPs or deletions wherein,
a SNP of the CBDAS gene is chosen from the group consisting of: “pos407”, “pos545”, “pos583”, “pos588”, “pos613”, “pos637”, “pos688” and “pos704”;
a SNP of the THCAS gene is chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases, position 153-156, and insertion of three bases, AAC, in position 755+3 in the CBDAS gene of high-THC type (drug-type) strains.

and wherein,

when the SNP of the CBDAS gene is:

G in position 407, the sample is a fiber-type variety;

A in position 407, the sample is a drug-variety;

G in position 545, the sample is a fiber-type variety;

C in position 545, the sample is a drug-variety;

A in position 583, the sample is a fiber-type variety;

C in position 583, the sample is a drug-variety;

T in position 583, the sample is a drug-variety;

C in position 588, the sample is a fiber-type variety;

T in position 588, the sample is a drug-variety;

A in position 613, the sample is a fiber-type variety;

G in position 613, the sample is a drug-variety;

C in position 637, the sample is a fiber-type variety;

G in position 637, the sample is a drug-variety;

T in position 688, the sample is a fiber-type variety;

A in position 688, the sample is a drug-variety;

C in position 704, the sample is a fiber-type variety;

G in position 704, the sample is a drug-variety;

when the SNP of the THCAS gene is:

C in position 136, the sample is a fiber-type variety;

G in position 136, the sample is a drug-variety;

C in position 137, the sample is a fiber-type variety;

T in position 137, the sample is a drug-variety;

A in position 154, the sample is a fiber-type variety;

G in position 154, the sample is a drug-variety;

C in position 221, the sample is a fiber-type variety;

T in position 221, the sample is a drug-variety;

T in position 269, the sample is a fiber-type variety;

A in position 269, the sample is a drug-variety;

G in position 287, the sample is a fiber-type variety;

C in position 287, the sample is a drug-variety;

C in position 300, the sample is a fiber-type variety;

T in position 300, the sample is a drug-variety;

T in position 355, the sample is a fiber-type variety;

A in position 355, the sample is a drug-variety;

C in position 383, the sample is a fiber-type variety;

T in position 383, the sample is a drug-variety;

A in position 385, the sample is a fiber-type variety;

G in position 385, the sample is a drug-variety;

A in position 409, the sample is a fiber-type variety;

T in position 409, the sample is a drug-variety;

G in position 412, the sample is a fiber-type variety;

A in position 412, the sample is a drug-variety;

G in position 418, the sample is a fiber-type variety;

A in position 418, the sample is a drug-variety;

A in position 424, the sample is a fiber-type variety;

G in position 424, the sample is a drug-variety;

T in position 494, the sample is a fiber-type variety;

A in position 494, the sample is a drug-variety;

T in position 505, the sample is a fiber-type variety;

C in position 505, the sample is a drug-variety;

C in position 612, the sample is a fiber-type variety;

T in position 612, the sample is a drug-variety;

A in position 678, the sample is a fiber-type variety;

G in position 678, the sample is a drug-variety;

A in position 699, the sample is a fiber-type variety;

T in position 699, the sample is a drug-variety;

T in position 744, the sample is a fiber-type variety;

G in position 744, the sample is a drug-variety;

T in position 749, the sample is a fiber-type variety;

A in position 749, the sample is a drug-variety;

G in position 763, the sample is a fiber-type variety;

T in position 763, the sample is a drug-variety;

A in position 862, the sample is a fiber-type variety;

G in position 862, the sample is a drug-variety;

G in position 864, the sample is a fiber-type variety;

A in position 864, the sample is a drug-variety;

C in position 869, the sample is a fiber-type variety;

T in position 869, the sample is a drug-variety.

In the present method, said sample can be any part of the plant, and in particular said sample is chosen from the group consisting of seeds, inflorescences (or flowers), leaves, roots, nodes, stem or stalk. The sample can be fresh or dried and the seeds can be peeled and fragmented using a pestle and mortar.

In the present method, the DNA extraction of step b. can be any extraction method commonly used in the laboratory, while said PCR of step c. is a technique known as “polymerase chain reaction” which is used to amplify DNA across several orders of magnitude and is carried out with a set of chosen and designed primers and/or primers and probes.

The primers and probes are chosen and designed on the desired SNP or deletion.

The isolated DNA can be evaluated for quantity and quality using spectrophotometric techniques or compared with a reference sample for any sample type (seeds, leaves . . . ).

The PCR analysis step c. can be carried out according to the preferred technique of the operator, and can be also a Taqman assay with labelled primers and probes.

To check the PCR product (fragment, or amplicon), the analysis step c. can be carried out by agarose gel electrophoresis, which allows size separation of the PCR products. The size(s) of PCR products is determined by comparison with a DNA ladder (a molecular weight marker), which contains DNA fragments of known size, run on the gel alongside the PCR products.

PCR product is purified and sequenced, analysis step d., sequence is purified by any method (enzymatic digestion, gel purification, column separation, ecc.) and processed by Capillary Electrophoresis, analysis step e. The obtained sequence is compared to a reference and SNPs and deletions/insertion are identified, analysis step f. This is a convenient method for verifying the SNPs and deletions/insertion, and is a preferred electrophoresis method.

In a preferred aspect, the electrophoresis is a capillary electrophoresis (CE), a family of electrokinetic separation methods performed in submillimeter diameter capillaries and in micro- and nanofluidic channels.

The sequencing step d. can be performed by capillary electrophoresis, by direct sequencing of PCR amplified fragment or in any technique that allows to identify the SNP and deletion.

In a further aspect the invention provides a kit for distinguishing between the fiber-type variety and the drug-type variety of Cannabis sativa by using one or more genetic markers chosen from the group consisting of:
SNPs of the CBDAS gene chosen from the group consisting of: “pos407”, “pos545”, “pos583”, “pos588”, “pos613”, “pos637”, “pos688” and “pos704”,
SNPs of the THCAS gene chosen from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases, position 153-156, and insertion of three bases, in position 755+3. in the CBDAS gene of high-THC type (drug-type) strains.

said kit comprising one or more sets of primers and/or probes and an instructions leaflet.

The present kit may be used according to the method of the invention, and may comprise further components necessary for DNA extraction from any plant sample or seed.

The diagnostic, genetic kit of the invention surprisingly facilitates early distinction of cannabis plants (i.e. before the maturity stage when the cannabinoid production starts) and selection of cannabis seeds according to their applications in the primary sector (cultivation of hemp for textiles, cosmetics, production of renewable energy) or pharmaceuticals (production of cannabinoids for therapeutic use), at the same time offering intelligence tools for controlling the illicit drug market.

One of the main advantages obtained by the present kit is that of being able to identify the plant variety from a sample such as a seed by analyzing only one genetic marker. Up to now this advantage was never obtained, nor previously described.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention.

### Example 1

### Experimental Cultivations and Chemical Analyses

10 different fiber-type varieties (Table 1a) have been chosen among a collection of hemp strains reproduced in the Institute of Agronomy, DIPROVES, “Università Cattolica del Sacro Cuore”, Piacenza, Italy. Seeds of fiber-type varieties have been sown in a randomized complete block design with four replicates. Sown and plants grown in an experimental field in Piacenza (North of Italy). 50 fiber-type plants were totally analyzed (5 each variety). Inflorescences were picked up at plant maturity stage, dried in oven at 40° C. for 48 h, then prepared for the chemical analysis according to Appendino et al. (2008).

Table 1. Main cannabinoid content of the plants (50 drug-types and 40 fiber-types) coming from the experimental cultivations and collected at the maturity stage. Values are expressed as percentage of dry weight of inflorescence.

TABLE 1a
Fiber-type varieties included in the study.

| | N. of | | | | | |
|---|---|---|---|---|---|---|
| NAME | plants | CBD* | CBD** | THC* | THC** | THC/CBD* |
| Santhica 27 | 5 | 0.00 | — | 0.00 | — | 0.0 |
| Carmagnola | 5 | 3.89 | 0.49 | 0.24 | 0.04 | 0.061 |
| Uso 31 | 5 | 0.24 | 0.13 | 0.03 | 0.02 | 0.125 |
| Ermes | 5 | 2.53 | 0.37 | 0.15 | 0.03 | 0.059 |
| Finola | 5 | 1.66 | 0.34 | 0.31 | 0.1 | 0.186 |
| Ermo | 5 | 0.01 | 0.01 | 0.00 | — | 0.0 |
| Futura 75 | 5 | 3.18 | 0.21 | 0.18 | 0.02 | 0.056 |
| C.S. | 5 | 3.91 | 0.36 | 0.24 | 0.02 | 0.079 |
| Tygra | 5 | 1.97 | 0.38 | 0.31 | 0.12 | 0.157 |
| Carmaleonte | 5 | 2.68 | 0.47 | 0.15 | 0.03 | 0.055 |

*Mean % of dry weight.
**SD % of dry weight.

Samples were crushed, cleaned from seeds and secondary stems, then finely milled with a spice grinder; a sub-sample (75 mg) was next extracted in 15 mL of methanol (reagent grade, Sigma-Aldrich) for 1 h at 50° C. in a ultrasonic bath, and centrifuged at 6000 g for 5 minutes. An aliquot of the extract was finally evaporated in oven at 50° C. for 2 h and then maintained at 120° C. for 120 minutes to achieve total cannabinoids decarboxylation.

Samples were re-dissolved in the initial volume of methanol and analyzed through an in-house method using liquid chromatography coupled to triple quadrupole tandem mass spectrometry (LC-MS/MS) via an electrospray ionization source. With this purpose, an Agilent 1200 series liquid chromatograph and an Agilent 6410A mass spectrometer were used. The reverse-phase chromatographic separation was achieved on a CORTECS C18 analytical column (2.7 μm, 150 mm×3 mm i.d.) equipped with a guard column (Waters, USA) and using a binary mobile phase system (solvent A: Milli Q water with 0.1% HCOOH, solvent B: MeOH with 0.1% HCOOH). The gradient was increased from 75% B to 90% B in 16 minutes, flow rate was 0.18 mL/min and column temperature was 45° C. Cannabinoids (THC and CBD) analysis was performed under multiple reaction monitoring and positive ionization mode.

Electrospray conditions were set as follows: capillary voltage 4000 V, heated vaporizer 300° C., nitrogen flow rate 8 L/min (18 psi), and nitrogen temperature 300° C. Each analyte was acquired using at least two tandem MS transitions, and daughter ions ratio was used for confirmatory purposes, thus providing with the required analytical specificity. All daughter ions were used both as qualifiers and quantifiers; the detailed LC-MS/MS tandem MS conditions are provided in table 1 Reference standards of each cannabinoid, ranging between 0.1 to 200 mg/kg in methanol, were used as external standards for calibration and quantification purposes.

11 different drug-type varieties (Table 1b) have been selected as suitable for the indoor experimental cultivation looking at declared plant features such as feminized, auto-flowering, height, flowering period and THC content, to include all different phenotypic features within the examined pool of samples. For drug-types, seeds were bought on Internet by different cannabis on-line shops. A half of seeds bought on-line, were sown indoor one pot per plant. 47 plants reached the stage of maturity under controlled environmental conditions.

TABLE 1b
Drug-type varieties included in the study.

| | N. of | | | | | |
|---|---|---|---|---|---|---|
| NAME | plants | CBD* | CBD** | THC* | THC** | THC/CBD* |
| 60 days | 3 | 0.22 | 0.19 | 6.97 | 3.64 | 32.17 |
| Wonder | | | | | | |
| BC God Bud | 11 | 0.20 | 0.08 | 6.40 | 3.89 | 32.00 |
| Chocolate | 4 | 0.96 | 0.77 | 5.42 | 2.75 | 5.67 |
| Kush | | | | | | |
| Chocolope | 3 | 0.44 | — | 5.65 | — | 12.84 |
| Flash | 2 | 0.14 | — | 7.20 | — | 51.43 |
| Babylon | | | | | | |
| Golden | 2 | 0.42 | 0.35 | 11.80 | 4.30 | 28.10 |
| Berry | | | | | | |
| Northern | 4 | 1.81 | 1.84 | 6.40 | 4.90 | 3.53 |
| Light | | | | | | |
| Shiatsu Kush | 8 | 0.40 | 0.19 | 6.10 | 3.39 | 15.44 |
| Skunk#11 | 4 | 2.15 | 1.34 | 2.08 | 0.77 | 0.97 |
| Star Ryder | 4 | 0.84 | 1.04 | 11.33 | 3.28 | 13.53 |
| Uk Werkle | 2 | 1.54 | 1.79 | 6.70 | 4.70 | 4.36 |

Chemical analyses were performed on leaves and inflorescences of dried plants after the indoor cultivation. About 100-150 milligrams of each homogenized samples were solubilized in chloroform solvent containing cholestane as internal standard. Samples were examined by gas chromatography (GC-FID) using a 7820A Agilent GC to identify and quantify THC and CBD percentage using a standardized analytical method. The column used was an Agilent HP-5 fused silica capillary of 30 meters length, 0.320 mm inner diameter and 0.25 μm film thickness (Agilent Technologies). The gas carrier (N2) flow was constant at 1 ml/min. 1 μl of each sample was injected into the GC-FID using a 5:1 split injection ratio. The injector temperature was 290° C. The column oven was programmed with an initial temperature of 200° C. for 0.5 minutes, and increased to 260° C. at a rate of 15 ° C./min, holding at 260° C. for 4 minutes. For the purpose of this study, it has been considered the percentage value of the following cannabinoids: THC as the main compound of drug-type varieties having psychoactive effects; CBD as the main compound of fiber-type varieties who shares with THC the same molecular precursor CBG.

### Example 2

### Isolation and Sequencing of DNA

Genetic analyses, performed on both seeds and fresh leaves of each fiber-type and drug-type varieties, have been preceded by the study of published THCA synthase (hereafter THCAS) and CBDA synthase (CBDAS) sequences and by the design of the related primers (Table 2). 50 seeds of fiber-type and 20 seeds of drug type varieties have been directly processed for the DNA extraction, amplification and sequencing.

In detail, the full length coding sequence of THCAS was sequenced using both external and internal primers available from literature (Kojoma et al., 2006; marked by an asterisk in Table 2). Primers for CBDAS were designed with Primer 3plus (www.bioinformatics.nl/cgi-bin/primer3plus) to be highly specific for the cannabidiolic acid synthase (avoiding amplification of THCAS) and with the aim to amplify both fiber-type and drug-type Cannabis. A couple of primers for each gene (THCAS and CBDAS) was used to generate the full length gene fragment while the other primers served as internal primers for sequencing reactions (as indicated in table 2). For each primer a BLASTn search was performed against GenBank (www.ncbi.gov) and the specific cannabis databases Comparative Genomics platform CoGe (http://genomevolution.org) and the Cannabis Genome Browser (http://genome.ccbr.utoronto.ca/). Only primers that have the 100% of homology to the corresponding gene were used for the following analysis.

The DNA extraction from fresh leaves was carried out using the specific commercial kit DNeasy Plant Mini Kit (Qiagen, Hilden, Germany) following the protocol provided by manufacturer while for the seeds, previously peeled and fragmented using pestle and mortar, the protocol was adapted (half volume of reagents was used). The isolated DNA was loaded on agarose gel at 1% and compared with a reference sample of DNA. The amount of extracted DNA was about 10 ng/μl and the quality was good for the analysis for both seeds and leaves (Table 2).

The amplification reaction, by the use of the Qiagen Multipex PCR Kit, was made in a final volume of 25 μl, containing 5 μl Multiplex PCR Master Mix, 0.8 μl of primers 10 μM each and 2 μl of DNA.

Amplification was performed in an Applied Biosystem (Foster City, Calif., USA) GeneAmp PCR System 9700 and PCR conditions were: preheating at 95° C. for 15 min, 30 cycles at 94° C. for 30 s, 57° C. for 90 s and 72° C. for 90 s with a final extension at 72° C. for 10 min.

The amplified products were loaded on 2% agarose gel in TBE 1×. After staining with ethidium bromide, the amplified products were photographed under UV light (254 nm). The amplified products were purified using Spin MSB PCRapace (Stratec molecular, Berlin, Germany). Sequencing was carried out using the BigDye Terminator v3.1 Cycle Sequencing Kit (LifeTechnologies, Carlsbad, Calif., USA) as follows: 4 μl reaction mix, primer 3.2 pmol and 2 μl of purified PCR product in 15 μl total volume. The sequences were purified with BigDye XTerminator Purification Kit and processed on ABI PRISM 3130 Genetic Analyzer (Applied Biosystem).

TABLE 2
Primers used for amplification and sequencing of THCAS and CBDAS genes.

[symbol]

| PRIMER | | | | AMPLICON | Tm | *from Kojoma et |
|---|---|---|---|---|---|---|
| NAME | SEQ ID NO: | SEQUENCE | APPLICATION | LENGHT (bp) | (° C.) | al, 2006 |
| THCAS-1-F | SEQ ID NO: 1 | GACTGAAGAAAAATGAATTGCTCAGCATTTTCC | Full length/ | 1665 | 50-55 | |
| THCAS-1-R* | SEQ ID NO: 2 | TCTATTTAAAGATAATTAATGATGATGCGGTGG | sequencing | | | primer b |
| THCAS-2-F | SEQ ID NO: 3 | ACTGAAGAAAAATGAATTGCTCAG | Full length/ | 1692 | 60 | |
| THCAS-2-R | SEQ ID NO: 4 | ATTTAAAGATAATTAATGATGATGCG | sequencing | | | |
| THCAS-3-F* | SEQ ID NO: 5 | CAAACTAGGTTGCTGTCCCATC | sequencing | | | primer c |
| THCAS-3-R* | SEQ ID NO: 6 | CGTCTTCTTCCCAGCTGATCT | sequencing | | | primer e |
| THCAS-1A-F* | SEQ ID NO: 7 | AGCTGGGAAGAAGACGGCTTTCTCA | sequencing | | | primer d |
| THCAS-1A-R | SEQ Id NO: 8 | CGCCAACAGTAGGGCAATACC | sequencing | | | primer f |
| THCAS-4-F* | SEQ ID NO: 9 | AATAACTCCCATATCCAAGCA | sequencing | | | primer g |
| THCAS-4-R* | SEQ ID NO: 10 | AGGACTCGCATGATTAGTTT | sequencing | | | primer h |
| THCAS-5-F | SEQ ID NO: 11 | CTGAAGATGAATTGCTCAG | sequencing | | | |
| THCAS-5-R | SEQ ID NO: 12 | ACAATTGGTCGTGTTGAGTGTAT | sequencing | | | |
| THCAS-6-F | SEQ ID NO: 13 | TCCAAGATTGGCGTATCTCAA | sequencing | 1635 | 53 | |
| CBDAS-1-F | SEQ ID NO: 14 | ATGAAGTGCTCAACATTCTC | Full length/ | | | |
| CBDAS-1-R | SEQ ID NO: 15 | TTAATGACGATGCCGTGG | sequencing | | | |
| CBDAS-2-F | SEQ ID NO: 16 | TCTCCTTTTGGTTTGTTTGCAAG | Full length/ | 1623 | 60 | |
| CBDAS-2-R | SEQ ID NO: 17 | CCGTGGAAGAGGTGGGATG | sequencing | | | |
| CRDAS-3-F | SEQ ID NO: 18 | CATGTCTCTCATATCCAAGG | sequencing | | | |
| CBDAS-1-R | SEQ ID NO: 19 | AAACAGTAGGGCAATACCCAG | sequencing | | | |
| CBDAS-4-F | SEQ ID NO: 20 | AATCATTGTAGCATGGAAAATTAG | sequencing | | | |
| CBDAS-4-R | SEQ ID NO: 21 | AGCACCGTTCTGCCCAGCG | sequencing | | | |
| CBDAS-5-F | SEQ ID NO: 22 | GGCAGAACGGTGCTTTCAAG | sequencing | | | |
| CBDAS-5-R | SEQ ID NO: 23 | TATTTGGATTCTTGGGATCATTTA | sequencing | | | |
| CBDAS6-R | SEQ ID NO: 24 | GAAGGAGTGACGATAACAAGT | sequencing | | | |
| CBDAS7-F | SEQ ID NO: 25 | AAGCATCTAAACTGGATT | sequencing | | | |
| CBDAS7-R | SEQ ID NO: 26 | TTAATGACGATGCCGTGGAAGAG | sequencing | | | |

[symbol]

### Example 3

### Investigating on Genetic Differences

Sequences obtained were edited and aligned against the following reference sequences (Table 3): Gene Bank ID KJ469374 (fiber-type CBDAS-cultivar Carmen) and KJ469378 (drug-type THCAS) from Weiblen et al., 2015. For each sample a consensus sequence was produced aligning all the sequences obtained, reverse and forward strands, to cover the entire region of the gene. The consensus was generated with the SeaView platform (Gouy et al., 2010). Heterozygous bases were indicated using the IUPAC symbols. Sequences were aligned and compared with the previously reported THCAS and CBDAS sequences. Sequences used in THCAS alignment were: AB212836, AB212829, AB212837 and AB212830 (from Kojoma et al., 2006) and AB057805 (from Sirikantaramas et al., 2004). Sequences used in CBDAS alignment were: AB292682 (from Taura et al., 2007), KP970864 and KP970857 (from Onofri et al., 2015) and KJ469375 (from Weiblen et al., 2015). All sequences were aligned using MUSCLE algorithm (Edgar, 2004), a tool of the MEGA6 software (Tamura et al., 2013).

The .fas files containing DNA sequences were converted in Comma Separated Values format using Fasta2excel online tool (http://users-birc.au.dk/biopv), then the resulting .csv files were imported in R version 3.0.2.

Selection of important loci was then performed as follows: each locus was treated as a categorical random variable, and used to predict the phenotype by means of univariate Firth (1993) penalized-likelihood logistic regression. Resulting p-values were adjusted for multiplicity using Benjamini & Hochberg (1995) correction, which was shown to be appropriate under this context of dependence of p-values in Farcomeni (2006, 2007).

The list of significant loci after adjustment was used to build a score, where 1 point was assigned if the locus coincided with that of the consensus sequence for drug strains, −1 points were assigned if the locus coincided with that of the consensus sequence for fiber strains, and 0 points otherwise. In order to obtain parsimonious and effective scores, we proceeded by comparing optimal scores based on one, two, three, up to the total number of significant loci. The optimal scores were obtained by weighting the loci so to maximize the Area Under the Receiver Operating Characteristics Curve (AUC), under the constraint that at most k weights were non-negative. The parameter k was varied from 1 to the total number of loci, also separately for deletions and SNPs.

TABLE 3
THCA synthase gene and CBDA synthase gene sequences

| ID | Reference | Name | Type | Gene |
|---|---|---|---|---|
| KJ469374 | Weiblen | Cannabis sativa cultivar | hemp/fiber | reference |
| | | Carmen cannabidiolic acid | | CBDAS |
| | | synthase (CBDA1) gene | | |
| KJ469376 | Weiblen | Cannabis sativa cultivar | marijuana/drug | reference |
| | | Skunk #1 cannabidiolic | | CBDAS |
| | | acid synthase (CBDA3) | | |
| | | pseudogene | | |
| KJ469375 | Weiblen | Cannabis sativa cultivar | marijuana/drug | CBDAS |
| | | Skunk #1 cannabidiolic | | |
| | | acid synthase (CBDA2) | | |
| | | pseudogene | | |
| AB292682 | Taura | Cannabis sativa CBDAS | hemp/fiber | CBDAS |
| | | mRNA for cannabidiolic | | |
| | | acid synthase | | |
| KP970864 | Onofri | Cannabis sativa isolate 6-5 | hemp/fiber | CBDAS |
| | | cannabidiolic acid synthase | | |
| | | mRNA | | |
| KP970857 | Onofri | Cannabis sativa isolate 5-1 | hemp/fiber | CBDAS |
| | | cannabidiolic acid synthase | | |
| KJ469380 | Weiblen | Cannabis sativa cultivar | hemp/fiber | reference |
| | | Carmen | | THCAS |
| | | tetrahydrocannabinolic acid | | |
| | | synthase (THCA2) gene | | |
| KJ469378 | Weiblen | Cannabis sativa cultivar | marijuana/drug | reference |
| | | Skunk #1 | | THCAS |
| | | tetrahydrocannabinolic acid | | |
| | | synthase (THCA1) gene | | |
| AB212836 | Kojoma | Cannabis sativa gene for | hemp/fiber | THCAS |
| | | tetrahydrocannabinolic acid | | |
| | | synthase, partial cds, | | |
| | | strain: 045 | | |
| AB212829 | Kojoma | Cannabis sativa gene for | marijuana/drug | THCAS |
| | | tetrahydrocannabinolic acid | | |
| | | synthase, partial cds, | | |
| | | strain: 001 | | |
| AB212837 | Kojoma | Cannabis sativa gene for | marijuana/drug | THCAS |
| | | tetrahydrocannabinolic acid | | |
| | | synthase, partial cds, | | |
| | | strain: 053 | | |
| AB212830 | Kojoma | Cannabis sativa gene for | hemp/fiber | THCAS |
| | | tetrahydrocannabinolic acid | | |
| | | synthase, partial cds, | | |
| | | strain: 005 | | |
| AB057805 | Sirikantaramas | Cannabis sativa mRNA for | marijuana/drug | THCAS |
| | | tetrahydrocannabinolic acid | | |
| | | synthase precursor, | | |
| | | complete cds | | |

### Example 4

### Discovery of Highly Predictive Value Markers as a Diagnostic Test

The comparison of sequences of cannabis samples analyzed in this study allowed us to identify highly reliable markers suitable to design a diagnostic test.

We have sequenced both THCAS and CBDAS genes, and we have selected the most discriminating loci among the 47 of THCA and 40 of CBDA observed as significant in both these synthase genes.

In particular, to design the diagnostic test able to discriminate fiber-type form drug-type varieties, we proceeded with the identification of loci with the highest predictive value starting from the following scores according to the type of mutation for the two investigated synthase genes.

a) A score based on deletion/insertion of CBDAS has been achieved by giving 1.1 points to the deletion in position 153, −1 point to the deletion in position 755 (the possible values of the score were then −1, 0, 0.1, 1.1). The AUC was in this case 99.87% (95% CI: 99.65%-100.00%) and the threshold 0 (the score>0 indicating assignment to drug-type) showed 100% sensitivity (95% CI: 100.00%-100.00%) and 95.56% specificity (95% CI: 88.37%-100.00%).

Therefore, CBDAS deletion/insertion is able to discriminate the two cannabis sub-groups.

b) A score based on SNPs of CBDAS gene was also evaluated and we found in this case an AUC 100% using any one of the following 8 loci: “pos407” “pos545” “pos583” “pos588” “pos613” “pos637” “pos688” “pos704”.

c) Finally, a score based only on SNPs of THCAS gene gave an AUC 100% using any one of the following 25 loci: “pos136” “pos137” “pos154” “pos221” “pos269” “pos287” “pos300” “pos355” “pos383” “pos385” “pos409” “pos412” “pos418” “pos424” “pos494” “pos505” “pos612” “pos678” “pos699” “pos744” “pos749” “pos763” “pos862” “pos864” “pos869”

Concerning the last two scores (b and c points) based on SNPs, only one locus among the 33 selected would be sufficient to discriminate the two sub-groups of varieties.

The score based on deletion/insertion of THCAS gene was discarded because of an AUC 75%.

The use of Benjamini & Hochberg (1995) correction guaranteed that the expected proportion of falsely detected SNPs was below 5%. The empirical results confirmed that all detected SNPs were highly discriminating between marijuana and hemp. Sensitivity and specificity were above 95% in correspondence of several thresholds. A sensitivity analysis showed that these outstanding results are not dependent on the scoring system used.

Therefore, by conducting experiments on almost 200 cannabis samples (fiber-type as well as drug-types, both plants and seeds, of identified varieties) and comparing the chemical profile of the plant at the stage of maturity with its genotype (by sequencing THCAS and CBDAS genes after specific primer design), we have found high predictive value markers able to discriminate fiber-type from drug-type varieties, thus distinguishing marijuana seeds from hemp seeds .

These genetic markers reached an AUC 100% even testing just CBDAS deletion jointly to one of 33 SNPs above mentioned.

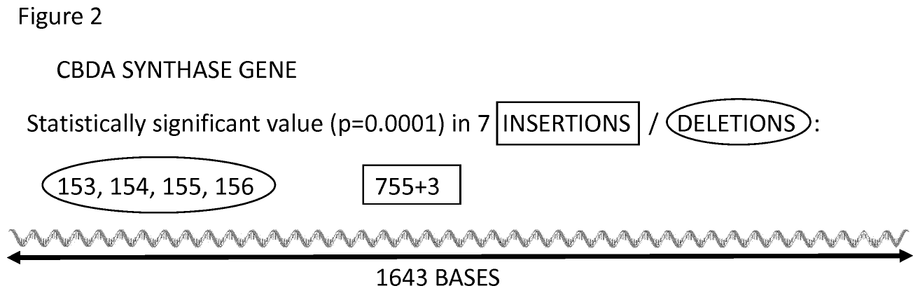

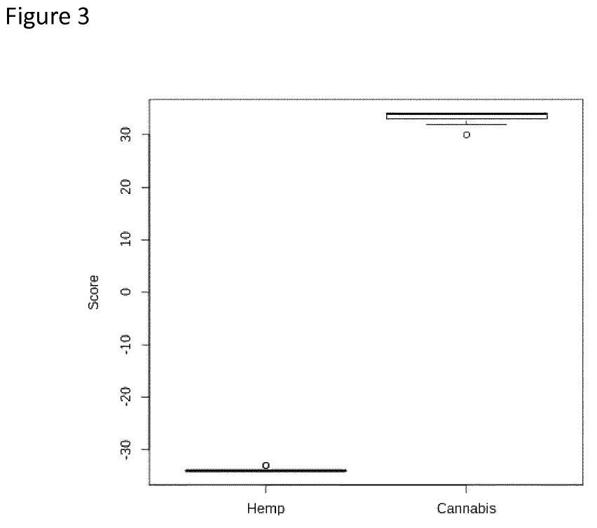

However, in order to make highly reliable the designed test and considering the possible low cost for its industrial realization, it would be desirable and recommended that this diagnostic, genetic test would be designed including CBDAS deletions/insertion together with the 33 identified SNPs (8 loci in CBDAS gene and 25 loci in THCAS gene). We call this score (d) throughout. The score achieves an AUC of 100%, sensitivity 100% (95% CI: 100.00%-100.00%) and specificity 100% (95% CI: 83.33%-100.00%) at the zero threshold. FIG. 2 shows a boxplot of the score values by plant type, and FIG. 3 shows the ROC curve.

### Example 5

### Use of the Markers in a Diagnostic Test

The genetic markers according to the present invention have allowed for the first time to surprisingly identify the phenotype of a cannabis plant, and in particular to distinguish the fiber-type (hemp) from the drug-type (marijuana) of Cannabis sativa by using one single marker.

Table 4 shows the precise nucleotides and the corresponding positions (locus, SNP) that allow to distinguish between the fiber-type from the drug-type (chemotypes) of the Cannabis sativa plant sample.

Up to now it has been seen that at least 4 SNPs are necessary to have a diagnostic distinction between the plant varieties (Rotherham, D. & Harbison, S. (2010)).

Apart from allowing the distinction of the varieties with only one marker, the markers of the present invention are distributed on the whole legth of the CBDAS and THCAS genes and can be selected according to the researcher's preferred position.

From the above description and the above-noted examples, the advantage attained by the product described and obtained according to the present invention are apparent.

The present invention therefore resolves the above-lamented problem with reference to the mentioned prior art, offering at the same time numerous other advantages, including allowing the development of a simple molecular assay which is capable of predicting the the phenotype of the cannabinoid plant, even from a seed, and therefore long before the plant reaches maturity.

TABLE 4
Correspondence table
CBDAS GENE

| | | | | | | | |
|---|---|---|---|---|---|---|---|
| G | G | A | C | A | C | T | C |
| pos | pos | pos | pos | pos | pos | pos | pos |
| 407 | 545 | 583 | 588 | 613 | 637 | 688 | 704 |
| A | C | C/T | T | G | G | A | G |

THCAS GENE

| C | C | A | C | T | G | C | T | C | A | A | G | G |
|---|---|---|---|---|---|---|---|---|---|---|---|---|
| pos | pos | pos | pos | pos | pos | pos | pos | pos | pos | pos | pos | pos |
| 136 | 137 | 154 | 221 | 269 | 287 | 300 | 355 | 383 | 385 | 409 | 412 | 418 |
| G | T | G | T | A | C | T | A | T | G | T | A | A |
| | A | T | T | C | A | A | T | T | G | A | G | C |
| | pos | pos | pos | pos | pos | pos | pos | pos | pos | pos | pos | pos |
| | 424 | 494 | 505 | 612 | 678 | 699 | 744 | 749 | 763 | 862 | 864 | 869 |
| | G | A | C | T | G | T | G | A | T | G | A | T |

### REFERENCES

Benjamini Y and Hochberg Y (1995) Controlling the False Discovery Rate: A Practical and Powerful Approach to Multiple Testing. J Royal Stat Soc. Series B, 57, 289-300

Edgar R C (2004) MUSCLE: multiple sequence alignment with high accuracy and high throughput. Nucleic Acids Res 32:1792-7.

Farcomeni, A. (2006) More Powerful Control of the False Discovery Rate under Dependence, Statistical Methods & Applications, 15, 43-73

Farcomeni, A. (2007) Some Results on the Control of the False Discovery Rate under Dependence, Scandinavian Journal of Statistics, 34, 275-297

Gouy M., Guindon S. & Gascuel O. (2010) SeaView version 4: a multiplatform graphical user interface for sequence alignment and phylogenetic tree building. Molecular Biology and Evolution 27(2):221-224.

Hillig, K. W. & Mahlberg, P. G. (2004). A chemotaxonomic analysis of cannabinoid variation in Cannabis (Cannabaceae). American Journal of Botany, 91, 966-75.

Kojoma M, Seki H, Yoshida S, Muranaka T. (2006) DNA polymorphisms in the tetrahydrocannabinolic acid (THCA) synthase gene in “drug-type” and “fiber-type” Cannabis sativa L. Forensic Sci Int. 2;159(2-3):132-40.

Rotherham, D. & Harbison, S. (2010). Differentiation of drug and non-drug Cannabis using a single nucleotide polymorphism (SNP) assay. Forensic Science International, 207, 1-3.

Sirikantaramas S, Morimoto S, Shoyama Y, Ishikawa Y, Wada Y, Shoyama Y, Taura F (2004) The gene controlling marijuana psychoactivity: molecular cloning and heterologous expression of Delta1-tetrahydrocannabinolic acid synthase from Cannabis sativa L. J Biol Chem 279: 39767-74

Tamura K, Stecher G, Peterson D, Filipski A, and Kumar S (2013) MEGA6: Molecular Evolutionary Genetics Analysis Version 6.0. Molecular Biology and Evolution 30: 2725-2729.

Taura, S., Morimoto, S. & Shoyama, Y. (1995). First direct evidence for the mechanism of D-1-tetrahydrocannabinolic acid biosynthesis. Journal of the American Chemical Society, 117, 9766-7.

Weiblen, G. D., Wenger, J. P., Craft, K. J., ElSohly, M. A., Mehmedic, Z., Treiber, E. L. and Marks, M. D. (2015), Gene duplication and divergence affecting drug content in Cannabis sativa. New Phytol, 208: 1241-1250

Yoshikai, K., Taura, T., Morimoto, S. & Shoyama, Y. DNA encoding cannabidiolate synthase, Patent in Japan JP 2000-78979A, 2001, DDBJ/EMBL/GenBank database accession numbers E55107.

1. A genetic marker for the discrimination/identification of the fiber-type variety from the drug-type variety of Cannabis sativa, wherein said genetic markers are:
SNPs of the CBDAS gene selected from the group consisting of: “pos545”, “pos588”, “pos407”, “pos583”, “pos613”, “pos637”, “pos688” and “pos704”,
SNPs of the THCAS gene selected from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”;
and
deletion of four bases, position 153-156, and
insertion of three bases, AAC, in position 755+3 in the CBDAS gene.

2. The genetic marker according to claim 1, wherein said genetic marker is selected from the group consisting of a SNP and a deletion of the CBDAS gene.

3. A method of distinguishing a sample of the
fiber-type variety of Cannabis sativa from the drug-type variety with the genetic marker according to claim 1, said method comprising
identifying at least one SNP of the CBDAS gene selected from the group consisting of: “pos545”, “pos588”, “pos407”, “pos583”, “pos613”, “pos637”, “pos688” and “pos704”,
SNPs of the THCAS gene selected from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”;
and
deleting four bases, position 153-156, and
inserting three bases, AAC, in position 755+3 in the CBDAS gene.

4. The method according to claim 3, wherein said sample from a Cannabis sativa plant is selected from the group consisting of a seeds, inflorescences (or flowers), leaves, roots, nodes, stem and stalk.

5. Method for discriminating the fiber-type variety from the drug-type variety of Cannabis sativa, comprising the steps of
a. providing a sample from a Cannabis sativa plant;
b. extracting the DNA from said sample;
c. conducting a PCR on the PCR sample of step b.;
d. sequencing the PCR product of step c.;
e. analyzing the sequence of said PCR product by electrophoresis
f. identifying at least one of the SNPs or deletions wherein,
a SNP of the CBDAS gene is selected chosen from the group consisting of: “pos545”, “pos588”, “pos407”, “pos583”, “pos613”, “pos637”, “pos688” and “pos704”;
a SNP of the THCAS gene is selected from the group consisting of:
“pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deleting four bases, position 153-156, and
inserting three bases, AAC, in position 755+3 in the CBDAS gene, and wherein,
when the SNP of the CBDAS gene is:
G in position 407, the sample is a fiber-type variety;
A in position 407, the sample is a drug-variety;
G in position 545, the sample is a fiber-type variety;
C in position 545, the sample is a drug-variety;
A in position 583, the sample is a fiber-type variety;
C in position 583, the sample is a drug-variety;
T in position 583, the sample is a drug-variety;
C in position 588, the sample is a fiber-type variety;
T in position 588, the sample is a drug-variety;
A in position 613, the sample is a fiber-type variety;
G in position 613, the sample is a drug-variety;
C in position 637, the sample is a fiber-type variety;
G in position 637, the sample is a drug-variety;
T in position 688, the sample is a fiber-type variety;
A in position 688, the sample is a drug-variety;
C in position 704, the sample is a fiber-type variety;
G in position 704, the sample is a drug-variety;
when the SNP of the THCAS gene is:
C in position 136, the sample is a fiber-type variety;
G in position 136, the sample is a drug-variety;
C in position 137, the sample is a fiber-type variety;
T in position 137, the sample is a drug-variety;
A in position 154, the sample is a fiber-type variety;
G in position 154, the sample is a drug-variety;
C in position 221, the sample is a fiber-type variety;
T in position 221, the sample is a drug-variety;
T in position 269, the sample is a fiber-type variety;
A in position 269, the sample is a drug-variety;
G in position 287, the sample is a fiber-type variety;
C in position 287, the sample is a drug-variety;
C in position 300, the sample is a fiber-type variety;
T in position 300, the sample is a drug-variety;
T in position 355, the sample is a fiber-type variety;
A in position 355, the sample is a drug-variety;
C in position 383, the sample is a fiber-type variety;
T in position 383, the sample is a drug-variety;
A in position 385, the sample is a fiber-type variety;
G in position 385, the sample is a drug-variety;
A in position 409, the sample is a fiber-type variety;
T in position 409, the sample is a drug-variety;
G in position 412, the sample is a fiber-type variety;
A in position 412, the sample is a drug-variety;
G in position 418, the sample is a fiber-type variety;
A in position 418, the sample is a drug-variety;
A in position 424, the sample is a fiber-type variety;
G in position 424, the sample is a drug-variety;
T in position 494, the sample is a fiber-type variety;
A in position 494, the sample is a drug-variety;
T in position 505, the sample is a fiber-type variety;
C in position 505, the sample is a drug-variety;
C in position 612, the sample is a fiber-type variety;
T in position 612, the sample is a drug-variety;
A in position 678, the sample is a fiber-type variety;
G in position 678, the sample is a drug-variety;
A in position 699, the sample is a fiber-type variety;
T in position 699, the sample is a drug-variety;
T in position 744, the sample is a fiber-type variety;
G in position 744, the sample is a drug-variety;
T in position 749, the sample is a fiber-type variety;
A in position 749, the sample is a drug-variety;
G in position 763, the sample is a fiber-type variety;
T in position 763, the sample is a drug-variety;
A in position 862, the sample is a fiber-type variety;
G in position 862, the sample is a drug-variety;
G in position 864, the sample is a fiber-type variety;
A in position 864, the sample is a drug-variety;
C in position 869, the sample is a fiber-type variety;
T in position 869, the sample is a drug-variety.

6. The method according to claim 3, wherein said sample from a Cannabis sativa plant is selected from the group consisting of a seeds, inflorescences (or flowers), leaves, roots, nodes, stem and stalk.

7. A kit for distinguishing between the fiber-type variety and the drug-type variety of Cannabis sativa by using one or more genetic markers selected from the group consisting of:
SNPs of the CBDAS gene selected from the group consisting of: “pos545”, “pos588”, “pos407”, “pos583”, “pos613”, “pos637”, “pos688” and “pos704”,
SNPs of the THCAS gene selected from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases of the CBDAS gene, position 153-156, and
insertion of three bases, AAC, in position 755+3 of the CBDAS gene, said kit comprising one or more sets of primers and/or probes and an instructions leaflet.

8. A kit for distinguishing between the fiber-type variety and the drug-type variety of Cannabis sativa by using one or more genetic markers selected from the group consisting of:
SNPs of the CBDAS gene selected from the group consisting of: “pos545”, “pos588”, “pos407”, “pos583”, “pos613”, “pos637”, “pos688” and “pos704”,
SNPs of the THCAS gene selected from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases of the CBDAS gene, position 153-156, and
insertion of three bases, AAC, in position 755+3 of the CBDAS gene, said kit comprising one or more sets of primers and/or probes and an instructions leaflet, for use in the method for discriminating the fiber-type variety from the drug-type variety of Cannabis sativa according to of claim 5.

9. A kit for distinguishing between the fiber-type variety and the drug-type variety of Cannabis sativa by using one or more genetic markers selected from the group consisting of:
SNPs of the CBDAS gene selected from the group consisting of: “pos545”, “pos588”, “pos407”, “pos583”, “pos613”, “pos637”, “pos688” and “pos704”,
SNPs of the THCAS gene selected from the group consisting of: “pos136”, “pos137”, “pos154”, “pos221”, “pos269”, “pos287”, “pos300”, “pos355”, “pos383”, “pos385”, “pos409”, “pos412”, “pos418”, “pos424”, “pos494”, “pos505”, “pos612”, “pos678”, “pos699”, “pos744”, “pos749”, “pos763”, “pos862”, “pos864” and “pos869”; and
deletion of four bases of the CBDAS gene, position 153-156, and
insertion of three bases, AAC, in position 755+3 of the CBDAS gene, said kit comprising one or more sets of primers and/or probes and an instructions leaflet, for use in a method of distinguishing a sample of the fiber-type variety of Cannabis sativa from the drug-type variety, wherein said sample from a Cannabis sativa plant is selected from the group consisting of a seeds, inflorescences (or flowers), leaves, roots, nodes, stem and stalk.


SEQUENCE LISTING (26 sequences)

SEQ ID NO: 1 - DNA, 33 bp, Cannabis sativa
gactgaagaaaaatgaattgctcagcattttcc

SEQ ID NO: 2 - DNA, 33 bp, Cannabis sativa
tctatttaaagataattaatgatgatgcggtgg

SEQ ID NO: 3 - DNA, 24 bp, Cannabis sativa
actgaagaaaaatgaattgctcag

SEQ ID NO: 4 - DNA, 26 bp, Cannabis sativa
atttaaagataattaatgatgatgcg

SEQ ID NO: 5 - DNA, 21 bp, Cannabis sativa
caaactggttgctgtcccatc

SEQ ID NO: 6 - DNA, 21 bp, Cannabis sativa
cgtcttcttcccagctgatct

SEQ ID NO: 7 - DNA, 25 bp, Cannabis sativa
agctgggaagaagacggctttctca

SEQ ID NO: 8 - DNA, 21 bp, Cannabis sativa
cgccaacagtagggcaatacc

SEQ ID NO: 9 - DNA, 21 bp, Cannabis sativa
aataactcccatatccaagca

SEQ ID NO: 10 - DNA, 20 bp, Cannabis sativa
aggactcgcatgattagttt

SEQ ID NO: 11 - DNA, 23 bp, Cannabis sativa
ctgaagaaaaatgaattgctcag

SEQ ID NO: 12 - DNA, 23 bp, Cannabis sativa
acaattggtcgtgttgagtgtat

SEQ ID NO: 13 - DNA, 21 bp, Cannabis sativa
tccaagattggcgtatctcaa

SEQ ID NO: 14 - DNA, 20 bp, Cannabis sativa
atgaagtgctcaacattctc

SEQ ID NO: 15 - DNA, 18 bp, Cannabis sativa
ttaatgacgatgccgtgg

SEQ ID NO: 16 - DNA, 23 bp, Cannabis sativa
tctccttttggtttgtttgcaag

SEQ ID NO: 17 - DNA, 19 bp, Cannabis sativa
ccgtggaagaggtgggatg

SEQ ID NO: 18 - DNA, 20 bp, Cannabis sativa
catgtctctcatatccaagg

SEQ ID NO: 19 - DNA, 21 bp, Cannabis sativa
aaacagtagggcaatacccag

SEQ ID NO: 20 - DNA, 24 bp, Cannabis sativa
aatcattgtagcatggaaaattag

SEQ ID NO: 21 - DNA, 19 bp, Cannabis sativa
agcaccgttctgcccagcg

SEQ ID NO: 22 - DNA, 20 bp, Cannabis sativa
ggcagaacggtgctttcaag

SEQ ID NO: 23 - DNA, 24 bp, Cannabis sativa
tatttggattcttgggatcattta

SEQ ID NO: 24 - DNA, 21 bp, Cannabis sativa
gaaggagtgacgataacaagt

SEQ ID NO: 25 - DNA, 18 bp, Cannabis sativa
aagcatctaaactggatt

SEQ ID NO: 26 - DNA, 23 bp, Cannabis sativa
ttaatgacgatgccgtggaagag